(19) United States
(12) Patent Application Publication
(10) Pub. No.: US 2022/0047448 A1
(43) Pub. Date: Feb. 17, 2022

(54) METHOD AND APPARATUS FOR PROVIDING MASSAGE TO STIMULATE GROWTH PLATE FOR PROMOTING GROWTH

(71) Applicant: BODYFRIEND CO., LTD., Seoul (KR)

(72) Inventors: Deok Hyun KONG, Seoul (KR); Soo Hyun CHO, Seoul (KR)

(21) Appl. No.: 17/275,267

(22) PCT Filed: Jan. 2, 2019
(86) PCT No.: PCT/KR2019/000011

§ 371 Date: Mar. 11, 2021

(30) Foreign Application Priority Data
Sep. 13, 2018 (KR) 10-2018-0109440

(51) Int. Cl. A61H 15/00; A61H 9/00

(52) U.S. Cl. CPC A61H 15/0078; A61H 9/0078; A61H 2201/0149; A61H 2015/0042; A61H 2201/164; A61H 2201/0192; A61H 2015/0014; A61H 2201/5023

(57) ABSTRACT

Provided are a method for providing a massage to stimulate a growth plate for promoting growth and a massage apparatus. The massage apparatus includes: a body data calculation unit for calculating length related information of at least a portion of a body part of a user; a growth plate stimulation massage pattern determination unit for determining a growth plate stimulation massage pattern on the basis of the calculated body length related information; and a growth plate stimulation massage providing unit for providing a massage on the basis of the growth plate stimulation massage pattern.

### TECHNICAL FIELD

The present invention relates to a method and an apparatus for providing a massage for stimulating a growth plate to promote growth, and more particularly, to a method and an apparatus configured to stimulate a growth plate to promote growth for providing a customized massage stimulating a growth plate according to physical conditions of a user.

### BACKGROUND ART

Generally, a physeal plate is a part of limb bone where length growth occurs and which is located on each of both ends of a bone and has a form in which cartilaginous tissue is interposed between bones. In the case of an embryo, all limb bones are formed of cartilage, and a central part of cartilage changes to a bone and gradually spreads toward both ends as the embryo grows. This part is referred to as a primary ossification center. Also, in a cartilage part on both ends of a limb bone which has not changed to bone yet, a part which autonomously changes to bone appears. This is referred to as a secondary ossification center. Simultaneously with vigorous cell division of cartilage cells located at a growth plate, cartilage gradually changes to a bone on a center and both ends of a bone. A cartilage part remaining therebetween becomes a growth plate. Here, while passing through puberty, all growth plates change to bones so that length growth is finished.

Recently, according to a result of research, factors having an influence on cell division of cartilage cells located on a growth plate include a genetic factor, nutrients, hormones, exercise, a mechanical stimulus, and the like. Among them, particularly, a growth degree of 70% or more depends on an acquired influential factor. Accordingly, as an embryo grows into adolescence, it is important to provide sufficient nutrition supply, adequate exercise, and a mechanical stimulus.

To this end, a variety of exercise methods and stretching devices for stimulating a growth plate of a limb bone highly related to growth in height have been provided.

As a related art, Korean Patent Registration No. 10-1675453 discloses a massage chair configured to recognize a body shape of a user seated in the massage chair and to determine a position of a growth acupuncture point of the user through comparison between general data on positions of acupuncture points according to age and gender and body shape data of the user so as to stimulate an acupuncture point part of the user.

### DISCLOSURE

### Technical Problem

The present invention is directed to providing a method and an apparatus for providing a massage stimulating a growth plate to promote growth in which a customized growth plate stimulating massage according to physical condition of a user is provided.

### Technical Solution

One aspect of the present invention provides a massage device configured to provide a growth plate stimulating massage promoting growth. The massage device includes a body data calculation portion configured to calculate length-related information of at least some body areas of a user, a growth plate stimulating massage pattern determination portion configured to determine a growth plate stimulating massage pattern on the basis of the calculated body-length-related information, and a growth plate stimulating massage providing portion configured to provide a massage on the basis of the growth plate stimulating massage pattern.

Another aspect of the present invention provides a method of providing a growth plate stimulating massage promoting growth. The method includes calculating length-related information of at least some body areas of a user, determining a growth plate stimulating massage pattern on the basis of the calculated body-length-related information, and providing a massage on the basis of the growth plate stimulating massage pattern.

### Advantageous Effects

A method and device for providing a growth plate stimulating massage promoting growth according to one embodiment of the present disclosure provide an effect of promoting physical growth of growing children or adolescents by stimulating and stretching knees and a spine on which growth plates are concentrated.

Also, the method and device for providing the growth plate stimulating massage promoting growth according to one embodiment of the present disclosure may provide a growth plate stimulating massage according to physical conditions of a user by adjusting a massage pattern on the basis of at least one of a height, imbalance between both leg lengths, and an upper-lower body proportion of the user.

Also, since the method and device for providing the growth plate stimulating massage promoting growth according to one embodiment of the present disclosure complexly consider two or more physical conditions as well as a single physical condition, it is possible to reflect more detailed physical conditions of the user in controlling the massage device.

In addition, since the method and device for providing the growth plate stimulating massage promoting growth according to one embodiment of the present disclosure may implement adjustment stepwise according to a variety of physical conditions with respect to one component of the device, it is possible to implement a massage device more optimized to the user.

### DESCRIPTION OF DRAWINGS

A variety of aspects will now be described with reference to the drawings. Here, like reference numerals refer to like elements. In the following embodiment, for purpose of description, a variety of particular details will be provided to give overall understanding of one or more aspects. However, it is apparent that such aspect(s) may be implemented without the particular details. In other examples, well-known structures and devices are illustrated as block diagrams to easily describe one or more aspects.

FIG. 1 is a view illustrating a massage device 1000 according to one embodiment of the present disclosure.

FIG. 2 is a view illustrating a main frame according to one embodiment of the present disclosure.

FIG. 3 is a view illustrating components of the massage device 1000 according to one embodiment of the present disclosure.

FIG. 4 is a front view illustrating the massage device 1000 according to one embodiment of the present disclosure.

FIG. 5 is a schematic side view illustrating some internal components of the massage device 1000 according to one embodiment of the present disclosure.

FIGS. 6A and 6B illustrate one example in which a position of a shoulder air cell 120 according to one embodiment of the present disclosure is adjusted according to physical conditions of a user.

FIGS. 7A to 7D illustrate one example in which a position of a leg massage portion 300 according to one embodiment of the present disclosure is determined on the basis of a length of user's legs.

FIGS. 8A to 8D illustrate one example in which a position of an upper leg module 320 according to one embodiment of the present disclosure is adjusted on the basis of physical conditions of the user.

FIGS. 9A and 9B illustrate one example of adjusting a depth degree to which a spine is pushed by a massage module 1700.

FIGS. 10A and 10B illustrate one example of comparison between shapes in which users with different heights are seated in the massage device according to one embodiment of the present disclosure.

FIGS. 11A and 11B are exemplary views illustrating a knee grow plate stimulating massage pattern according to one embodiment of the present disclosure.

FIG. 12 is an exemplary view illustrating a spine growth plate stimulating massage pattern according to one embodiment of the present disclosure.

FIGS. 13A and 13B are exemplary views illustrating a spine and knee grow plate stimulating massage pattern according to one embodiment of the present disclosure.

FIG. 14 is a flowchart illustrating a growth plate stimulating massage providing method according to one embodiment of the present disclosure.

### BEST MODES

According to one embodiment of the present disclosure, there is provided a massage device configured to provide a growth plate stimulating massage promoting growth. The massage device includes a body data calculation portion configured to calculate length-related information of at least some body areas of a user, a growth plate stimulating massage pattern determination portion configured to determine a growth plate stimulating massage pattern on the basis of the calculated body-length-related information, and a growth plate stimulating massage providing portion configured to provide a massage on the basis of the growth plate stimulating massage pattern.

Also, there is provided a method of providing a growth plate stimulating massage promoting growth. The method includes calculating length-related information of at least some body areas of a user, determining a growth plate stimulating massage pattern on the basis of the calculated body-length-related information, and providing a massage on the basis of the growth plate stimulating massage pattern.

### Modes of the Invention

The aspects, features, and advantages of the present disclosure will be more apparent from the following embodiment in relation to the attached drawings. The following particular structure and functional descriptions are exemplified to describe an embodiment according to the concept of the present disclosure. Embodiments according to the concept of the present disclosure may be implemented in a variety of shapes and should not be construed as being limited to the embodiments described in the specification or application.

Since a variety of modifications may be made in the embodiments according to the concept of the present disclosure and the embodiments may have a variety of shapes, particular embodiments will be illustrated in the drawings and described in detail in the specification or application. However, these are not intended to limit the embodiments according to the concept of the present disclosure to a particularly disclosed form and it should be understood that the embodiments according to the concept of the present disclosure include all changes, equivalents, and substitutes included within the concept and technical scope of the present disclosure.

The terms such as first, second, and/or the like may be used to describe a variety of components, and the components should not be limited to the terms. The terms are used only for distinguishing one component from another. For example, a first component may be referred to as a second component, and similarly, the second component may be referred to as the first component without departing from the scope of the concept of the present disclosure.

When it is stated that one component is “connected” or “joined” to another component, it should be understood that the one component may be directly connected or joined to the other component but still another component may be present therebetween. On the other hand, when it is stated that one component is “directly connected” or “directly joined” to another component, it should be understood that still another component is not present therebetween. Other expressions for describing a relationship between components, that is, “between,” “directly between,” “adjacent to,” “directly adjacent to,” and the like should also be equally construed.

The terms used herein are used merely for describing particular embodiments and are not intended to limit the present disclosure. Singular expressions, unless clearly defined otherwise in context, include plural expressions. Throughout the specification, the terms “comprise,” “have,” or the like are used herein to specify the presence of implemented features, numbers, stages, operations, elements, components, or combinations thereof but do not preclude the presence or addition of one or more other features, numbers, stages, operations, elements, components, or combinations thereof.

Unless otherwise defined, all terms used herein including technical or scientific terms have the same meanings as those generally understood by one of ordinary skill in the art. The terms as defined in generally used dictionaries should be construed as having meanings which coincide with contextual meanings of related art and are not to be understood as ideal or excessively formal meanings unless clearly defined.

In the specification, an actuator means a component capable of providing a driving force. For example, the actuator may include an actuator, a linear actuator, an electronic actuator, a direct current (DC) actuator, an alternating current (AC) actuator, a linear actuator, an electric actuator, and the like but is not limited thereto.

In the specification, a spiral rod means a linear member having a spiral groove and may be implemented using a metal material. For example, the spiral rod may include a cylindrical bar having a spiral groove on a surface thereof. Also, the spiral rod may include a metal lead screw.

According to one embodiment of the present disclosure, a massage device may refer to a massage device including a body massage portion and a leg massage device.

The terms “information” and “data” used herein may be interchangeable with each other.

A sound source in the present disclosure may include a variety of types of music such as classic music, jazz music, instrumental music, pop music, and the like, natural sounds such as a sound of water, a birdcall, and the like, and an arbitrary audio type of information including a binaural beat and the like. Also, in the specification, a binaural beat may mean audio information having a particular form capable of adjusting a brainwave.

Hereinafter, embodiments of the present disclosure will be described in more detail with reference to the attached drawings.

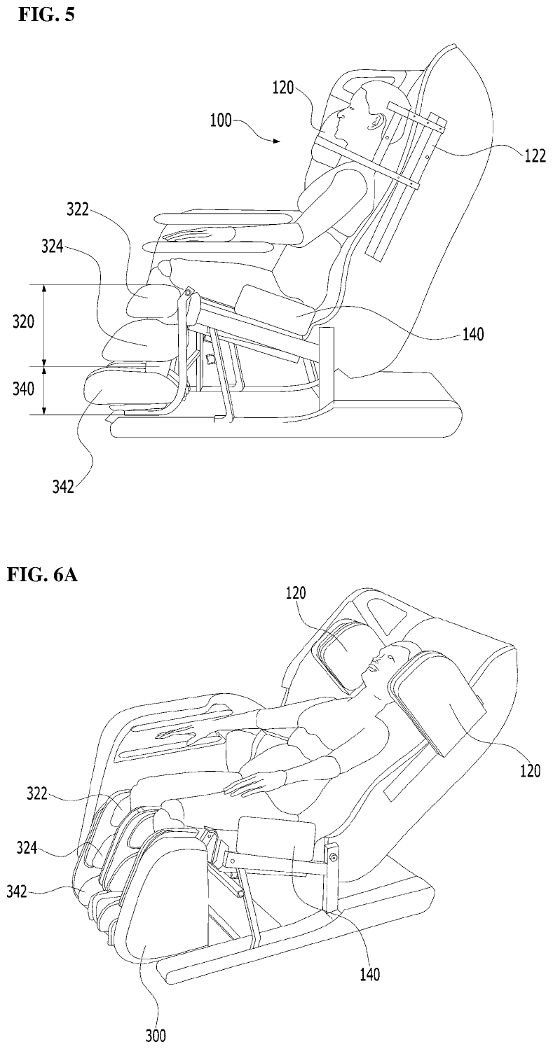

FIG. 1 is a view illustrating a massage device 1000 according to one embodiment of the present disclosure, FIG. 4 is a front view illustrating the massage device 1000 according to one embodiment of the present disclosure, and FIG. 5 is a schematic side view illustrating some internal components of the massage device 1000 according to one embodiment of the present disclosure.

The massage device 10 according to one embodiment of the present disclosure may include a body massage portion 100 configured to form an area for accommodating at least a part of a body of a user and to massage the body of the user and a leg massage portion 300 configured to massage legs of the user.

The body massage portion 100 may provide a massage to at least a part of the body of the user. The body massage portion 100 may include a massage module 1700 configured to provide a massage function on at least a part of the body of the user, an audio output module 1600 configured to provide audio output having an arbitrary form toward the user, a main frame 1100 configured to form a framework of the body massage portion 100, and a user input portion 1800 configured to receive input having an arbitrary form from the user.

The above-described components included in the body massage portion 100 are merely an exemplary embodiment, and the body massage portion 100 may include a variety of components in addition to the above-described components.

Also, a shape and a structure of the massage device 1000 shown in FIG. 1 are merely an example, and a variety of shapes of the massage device 1000 may be included within the scope of the present disclosure without departing from the scope of the present disclosure which is defined by the claims.

The body massage portion 100 may form a space having an arbitrary shape for accommodating the user. The body massage portion 100 may include a space having a shape corresponding to a shape of the body of the user. For example, as shown in FIG. 1, the body massage portion 100 may be implemented as a sitting type capable of accommodating an entirety or a part of the body of the user.

A part of the body massage portion 100 which comes into contact with the ground may include an arbitrary material for increasing a frictional force or an arbitrary member (for example, a skid-proof pad and the like) for increasing a frictional force and may include wheels configured to reinforce mobility of the massage device 1000.

At least a part of the body massage portion 100 is slidable. For example, when the body massage portion 100 starts massage, at least a part of the body massage portion 100 is slidable forward. Also, the body massage portion 100 may be tilted backward. As a result, the body massage portion 100 may provide a massage while being titled backward.

According to one embodiment of the present disclosure, the massage device 1000 may include at least one air cell.

In particular, the massage device 1000 according to embodiments of the present disclosure may include a shoulder air cell 120 corresponding to a shoulder part of a user, a pelvis air cell 140 corresponding to a pelvis part, an arm air cell corresponding to an arm massage portion, an upper air cell 322 provided on the leg massage portion 300, an intermediate air cell 324, a lower air cell 342, and a heel air cell 344 but is not limited thereto and may include air cells on a variety of parts of the massage device 1000.

The massage device 1000 may include an air supply portion, and the air supply portion may supply air to the air cells so as to inflate the air cells. The air supply portion may be located inside the body massage portion 100 and may be located in the leg massage portion 300. Also, the air supply portion may be located outside the massage device 1000.

The leg massage portion 300 may provide the user with a leg massage.

For example, the leg massage portion 300 may include a calf massage portion configured to massage calves of the user and/or a foot massage portion configured to massage feet of the user.

The leg massage portion 300 may be adjustable in length according to physical features of the user. For example, when a tall user uses the massage device 1000, since calves of the user are long, it is necessary to increase a length of the leg massage portion 300. Also, when a short user uses the massage device 1000, since calves of the user are short, it is necessary to decrease the length of the leg massage portion 300. Accordingly, the leg massage portion 300 may provide the user with a leg massage customized according to a height of the user.

Particularly, referring to FIGS. 4 and 5, the leg massage portion 300 may include the upper leg module 320 and the lower leg module 340. Also, each of the upper leg module 320 and the lower leg module 340 may include a vertically movable structure. The upper leg module 320 may include the upper air cell 322 and the intermediate air cell 324 thereinside, and the lower leg module 340 may include the lower air cell 342 thereinside. Air may be injected into or discharged from the upper air cell 322, the intermediate air cell 324, and the lower air cell 342 independently according to a massage pattern.

On the basis of a human body seated in the massage device 1000, the upper air cell 322 may correspond to both sides of a bottom end of a femur, the intermediate air cell 324 may correspond to outsides of a fibula and a tibia, and the lower air cell 342 may correspond to an ankle.

Also, for a user such as a child and the like who has relatively small feet, a heel air cell (not shown) may be provided on a part corresponding to a heel. The heel air cell may be provided in order to fix a foot not to be detached or slipping out from the lower leg module 340 during extension.

The massage module 1700 may be provided inside the body massage portion 100 so as to provide the user accommodated in the body massage portion 100 with a dynamic stimulus having an arbitrary form. As shown in FIG. 1, the massage module 1700 may move along a main frame 1100 provided inside the body massage portion 100.

For example, the main frame 1100 of the body massage portion 100 may include a rack gear, and the massage module 1700 may provide dynamic stimuli to a variety of parts of the body of the user while moving along the rack gear. The massage module 1700 may include a ball massage unit or a roller massage unit but is not limited thereto.

The main frame 1100 forms a framework of an interior of the body massage portion 100 and may be implemented using a metal material, a plastic material, or the like. For example, the main frame 1100 may be implemented using iron, an alloy, steel, or the like but is not limited thereto and may be implemented using a variety of hard materials.

According to one embodiment of the present disclosure, the massage device 1000 may include the audio output module 1600. The audio output module 1600 may be provided in a variety of positions. For example, the audio output module 1600 may include a plurality of output units such as a top end audio output unit disposed at a top end of a seat portion which comes into contact with the user, front audio output units attached to front ends of left and right arm massage portions of the seat portion, and/or rear audio output units attached to rear ends of the arm massage portions but is not limited thereto. In this case, the audio output module 1600 may provide a stereophonic sound such as 5.1 channel surround sound but is not limited thereto.

According to one embodiment of the present disclosure, the user may control the massage device 1000 using a massage device control device 2000. The massage device control device 2000 may be connected to the massage device 1000 through wired communication and/or wireless communication.

The massage device control device 2000 may include a remote controller, a cellular phone, personal digital assistant (PDA), and the like but is not limited thereto and may include a variety of electronic devices capable of being connected to the massage device 1000 through wired or wireless communication.

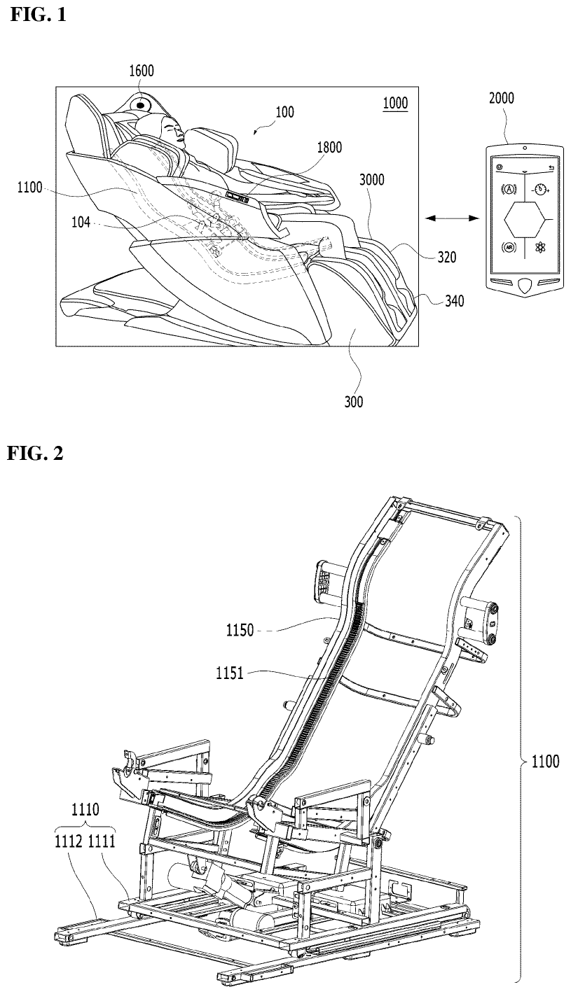

FIG. 2 is a view illustrating the main frame according to one embodiment of the present disclosure.

According to one embodiment of the present disclosure, the main frame 1000 may include an upper frame 1150 on which the massage module 1700 is provided and a base frame 1110 configured to support the upper frame 1150.

A rack gear 1151 may be provided on at least a part of the upper frame 1150. The rack gear 1151 is a member configured to guide vertical movement of the body massage module 1700 and may include a plurality of valley portions and a plurality of ridge portions.

According to one embodiment of the present disclosure, the rack gears 1151 may be provided on both sides of the upper frame 1150 to face each other, and the body massage module 1700 may move along the rack gear 1151.

For example, the body massage module 1700 may include a gear to be engaged with the rack gear 1151. The gear is rotated by an actuator provided in the body massage module 1700 so that the body massage module 1700 may move upward or downward.

The rack gear 1151 may be implemented using a metal material or a plastic material. For example, the rack gear 1151 may be implemented using iron, steel, an alloy, reinforced plastics, a melamine resin, a phenolic resin, and the like but is not limited thereto.

The upper frame 1150 may have a variety of shapes. For example, the upper frame 1150 may be divided, according to a shape, into an S frame, an L frame, an S&L frame, and a double S&L frame but is not limited thereto.

The S frame means at least a partial frame of the upper frame 1150 which includes a shape that is curved like the letter S. The L frame means at least a partial frame of the upper frame 1150 which includes a shape that is bent like the letter L. The S&L frame means a frame which includes both the shape curved like the letter S and the shape bent like the letter L. The double S&L frame means a frame including a shape bent like the letter L and a shape of two parts curved like the letter S.

The base frame 1110 means a part which supports the upper frame 1150 of the main frame 1100 and comes into contact with the ground. The base frame 1110 may include an upper base frame 1111 and a lower base frame 1112.

The upper base frame 1111 may support the upper frame 1150, and the lower base frame 1112 may come into contact with the ground. Also, the upper base frame 1111 may be located to come into contact with the lower base frame 1112.

According to one embodiment of the present disclosure, the upper base frame 1111 may move along the lower base frame 1112. For example, the upper base frame 1111 may slide forward or backward along the lower base frame 1112. In this case, the upper frame 1150 may be connected to the upper base frame 1111 and move according to movement of the upper base frame 1111.

For example, when the upper base frame 1111 moves forward, the upper frame 1150 may also move forward. When the upper base frame 1111 moves backward, the upper frame 1150 may also move backward. Accordingly, the body massage portion 100 may be allowed to be slidable.

In detail, to allow the upper base frame 1111 to move, a moving wheel may be provided below the upper base frame 1111. Also, a guide member capable of guiding the moving wheel may be provided above the lower base frame 1112. The moving wheel provided at the upper base frame 1111 may move along the guide member provided at the lower base frame 1112 so as to allow the upper base frame 1111 to move forward or backward.

According to another embodiment of the present disclosure, the massage device 1000 may not provide a sliding function. In this case, the base frame 1110 may not be separated into upper and lower frames.

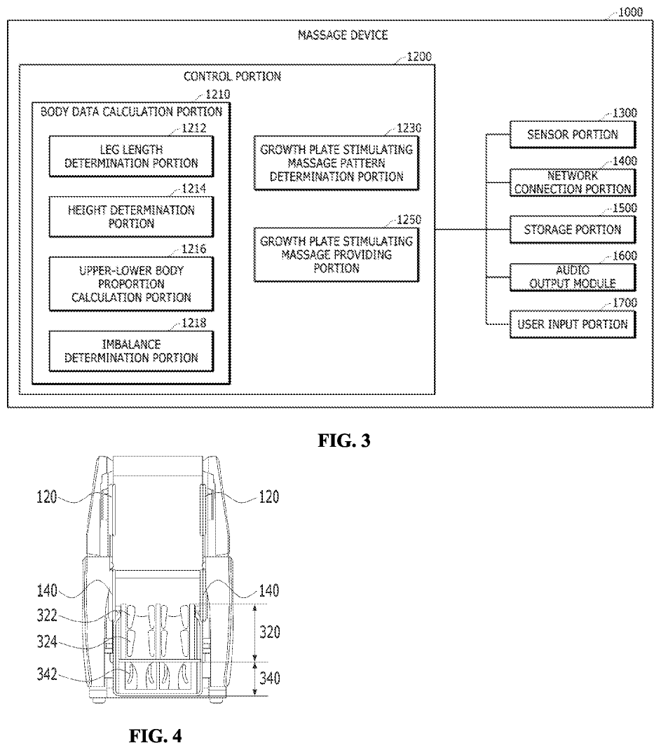

FIG. 3 is a view illustrating components of the massage device 1000 according to one embodiment of the present disclosure.

According to one embodiment of the present disclosure, the massage device 1000 may include at least one of a control portion 1200, a sensor portion 1300, a network connection portion 1400, a storage portion 1500, an audio output module 1600, and a user input portion 1700.

The control portion 1200 may control operations of the massage device 1000. The control portion 1200 may be implemented using one processor or be implemented using a plurality of processors. When the control portion 1200 is implemented using a plurality of processors, at least some of the plurality of processors may be located at a physically spaced distance. Also, the control portion 1200 is not limited thereto and may be implemented using a variety of methods.

According to one embodiment of the present disclosure, the control portion 1200 may control operations of the massage device 1000. For example, the massage device 1000 may include a plurality of actuators, and the operations of the massage device 1000 may be controlled by controlling operations of the plurality of actuators. For example, the massage device 1000 may include at least one of an actuator configured to move the massage module 1700, at least one actuator included in the massage module, a back angle actuator, a leg angle actuator, a foot massage actuator, a leg length adjustment actuator, and a sliding actuator. The control portion 1200 may control the operations of the massage device 1000 by controlling the same.

The actuator configured to move the massage module is an actuator configured to allow the massage module 1700 to move vertically. The massage module 1700 may move along the rack gear due to an operation of the actuator configured to move the massage module 1700.

The back angle actuator is an actuator configured to adjust an angle of a part of the massage device 1000 which comes into contact with the back of the user. A back angle of the massage device 1000 may be adjusted by an operation of the back angle actuator.

The leg angle actuator is an actuator configured to adjust an angle of the leg massage portion 300 of the massage device 1000. An angle between the leg massage portion 300 and the body massage portion 100 may be adjusted by an operation of the leg angle actuator.

The foot massage actuator refers to an actuator configured to operate the foot massage module included in the leg massage portion 300. The massage device 1000 may provide the user with a foot massage utilizing the foot massage actuator.

The massage module 1700 may include at least one actuator, and the control portion 1200 may provide a variety of massage operations by operating the at least one actuator. For example, the control portion 1200 may provide a beating massage, a kneading massage, and the like by operating the at least one actuator included in the massage module 1700 but is not limited thereto and may provide a variety of massage operations.

The leg length adjustment actuator refers to an actuator configured to adjust a length of the leg massage portion 300. For example, the control portion 1200 may adjust the length of the leg massage portion 300 according to the user by utilizing the leg length adjustment actuator so that the user may receive a massage adequate for his or her body shape.

The sliding actuator enables a sliding operation of the massage device 1000. For example, a horizontal upper base frame 1114a may move forward or backward due to an operation of the sliding actuator so that the upper frame connected to the horizontal upper base frame 1114a may also move forward or backward.

The sensor portion 1300 may obtain a variety of pieces of information using at least one sensor. The sensor portion 1300 may be provided as a sensor utilizing means for measuring a pressure, potential, optics, and the like. For example, the sensor may include a pressure sensor, an infrared sensor, a light emitting diode (LED) sensor, a touch sensor, and the like and is not limited thereto.

Also, the sensor portion 1300 may include a biometrics obtaining sensor. The biometrics obtaining sensor may obtain fingerprint information, face information, voice information, iris information, weight information, electrocardiogram information, and the like but is not limited thereto and may include a variety of pieces of biometric information.

According to another embodiment of the present disclosure, the massage device 1000 may sense a contact area and/or a contact position with the user using the sensors. Also, the massage device 1000 may obtain shoulder position information of the user through the sensor portion 1300. Also, the massage device 1000 may provide a customized massage based on the obtained information. For example, when the massage device 1000 provides a shoulder massage, the massage device 1000 may recognize the shoulder position of the user on the basis of the information obtained using the sensor portion 1300 and provide the user with a shoulder massage according to a recognition result.

The user input portion 1800 may receive a command related to controlling the operation of the massage device 1000 from the user, and the user input portion 1800 may be implemented to have a variety of forms. For example, the user input portion 1800 may be provided in the body massage portion 100 and provided in the leg massage portion 300 but is not limited thereto.

The massage device 1000 may obtain a variety of commands from the user through the user input portion 1800. For example, the massage device 1000 may receive an arbitrary command related to a selection of the massage module, a selection of a massage type, a selection of a level of massage intensity, a selection of massage time, a selection of a massage part, a selection of a position and an operation of the body massage portion 100, a selection with respect to turning power of the massage device 1000 on and off, a selection with respect to whether a warming function is performed, a selection related to reproducing a sound source, and the like but is not limited thereto.

Particularly, the massage device 1000 according to embodiments of the present disclosure may provide a user interface configured to allow a user to select a growth plate stimulating massage mode. For example, a variety of medical massage modes related to physical improvement may be listed and a screen configured to allow the user to select a growth plate stimulating massage mode among the medical massage modes may be provided through the massage device control device 2000. The medical massage modes may include at least one of a concentration mode, a meditation mode, a recovery mode, a stretching mode, a sleeping mode, a vitality mode, a golf mode, a hip-up mode, an examinee mode, a weightless mode, and a growth mode.

When the user selects the growth mode, a guide message such as “User's physical conditions will be measured and a customized growth plate stimulating massage will be provided” and a description on an effect provided by the growth plate stimulating massage mode may be provided through the massage device control device 2000 or a speaker. In addition, information on a result of measuring user's physical information may be provided. For example, the physical information measuring result may include information on a height, a leg length, and an upper-lower body proportion of the user as well as information on whether left and right legs are asymmetrical or the like. The above interface, providing of information, and the like are merely one example and the present disclosure may not be limited thereto.

According to another embodiment of the present disclosure, the user input portion 1800 may include hot-key type buttons, a selection button for implementing a selection, cancellation, or input of direction, and/or the like according to a preset user setting function, an autonomously preset function, or the like.

The user input portion 1800 may be implemented using a key pad, a dome switch, a touch pad (static pressure/static electricity), a jog wheel, a jog switch, and the like but is not limited thereto. Also, the user input portion 1800 may obtain a command through an utterance of the user on the basis of voice recognition art.

According to one embodiment of the present disclosure, the user input portion 1800 may include a display configured to display an operation state of the massage device 1000, a current state of the user, or the like. In this case, the display may include at least one of a liquid crystal display (LCD), a thin film transistor LCD (TFT LCD), an organic light emitting diode (OLED), a flexible display, and a three-dimensional (3D) display but is not limited thereto.

The audio output module 1600 may output arbitrarily-formed audio to the user. For example, the audio output module 1600 may stimulate the brain of the user by outputting a sound source and/or a binaural beat optimized to a massage pattern provided by the massage device 1000. The audio output module 1600 may output a voice signal which is received through a network (not shown) or stored in an internal/external storage medium (not shown). For example, the audio output module 1600 may output the sound source under control of a user terminal 2000 through network connection (for example, Bluetooth connection and the like) with the user terminal 2000. Also, the audio output module 1600 may output an arbitrarily-formed sound signal generated in relation to the operation of the massage device 1000.

The massage device 1000 according to one embodiment of the present disclosure may include the network connection portion 1400. The network connection portion 1400 may perform communication with the modules inside the massage device 1000, an external massage device, and/or the user terminal 2000 through an arbitrarily-formed network. The network connection portion 1400 may include a wired/wireless connection module for network connection. As a wireless connection art, for example, wireless local area network (WLAN), Wi-Fi, wireless broadband (WiBro), world interoperability for microwave access (WiMAX), high speed downlink packet access (HSDPA), and the like may be used. As a wired connection art, for example, x digital subscriber line (xDSL), fibers to the home (FTTH), power line communication (PLC), and the like may be used. Also, the network connection portion may include a short-range communication module so as to transmit or receive data with an arbitrary device/terminal located at a short distance. For example, as a short range communication art, Bluetooth, radio frequency identification (RFID), infrared data association (IrDA), ultra wideband (UWB), ZigBee, and the like may be used but the present disclosure is not limited thereto.

A storage portion 1500 may store a variety of pieces of information related to the massage device 1000. For example, the storage portion 1500 may include massage control information and include personal authentication information but is not limited thereto.

Particularly, the storage portion 1500 according to the present disclosure may include information on at least one of a knee growth plate stimulating massage pattern, a spine growth plate stimulating massage pattern, and a knee/spine growth plate stimulating massage pattern as the growth plate stimulating massage pattern.

The information on each growth plate stimulating massage pattern may include at least one of (1) a total massage time provided to the user according to a particular mode, (2) a massage type indicating a type of dynamic stimulation provided by the massage device, (3) a massage part or massage position according to a particular mode, (4) a massage level indicating intensity of the dynamic stimulation provided by the massage device, and/or (5) an operating method and an operating sequence of the massage device.

The storage portion 1500 may be implemented using a non-volatile storage medium capable of continuously storing arbitrary data. For example, the storage portion 1500 may include not only a disc, an optical disk, and a magneto-optical storage device but also a flash memory and/or a storage device based on a battery-backup memory but is not limited thereto.

Also, the storage portion 1500 may include a memory. The memory is a main storage device like a random-access memory (RAM) such as a dynamic RAM (DRAM), a static RAM (SRAM), and the like, which is directly accessed by a processor and may mean a volatile storage device in which stored information is instantaneously erased when power is turned off but is not limited thereto. The memory may be operated by the control portion 1200.

The control portion 1200 may include a body data calculation portion 1210, a growth plate stimulating massage pattern determination portion 1230, and a growth plate stimulating massage providing portion 1250.

The body data calculation portion 1210 may calculate length-related information of at least a part of a user's body, the growth plate stimulating massage pattern determination portion 1230 may determine the growth plate stimulating massage pattern on the basis of the calculated body length-related information, and the growth plate stimulating massage providing portion 1250 may provide a massage on the basis of the growth plate stimulating massage pattern.

The body data calculation portion 1210 may include at least one of a leg length determination portion 1212, a height determination portion 1214, an upper-lower body proportion calculation portion 1216, and an imbalance determination portion 1218.

The leg length determination portion 1212 may obtain a heel position of the user seated in the massage device 1000 through a sensing means and may determine a leg length of the user on the basis thereof. For example, the leg massage portion 300 may be moved to unfold legs mounted on the leg massage portion 300 to be parallel to the ground and the heel position may be measured while a user's legs are unfolded. Since length information of a seat region of the massage device 1000 is already known, the leg length of the user may be determined on the basis thereof.

Here, in determining the leg length of the user, the leg length determination portion 1212 may further use stored information obtained by previously numerating information on a relationship between leg lengths measured while a plurality of people stand upright and leg lengths measured while the plurality of people are seated in the massage device 1000.

As one embodiment, the leg length determination portion 1212 may determine each of left and right leg lengths. To this end, the leg length determination portion 1212 may obtain a left heel position and determine the left leg length on the basis thereof and obtain a right heel position and determine the right leg length on the basis thereof.

The imbalance determination portion 1218 may calculate a difference between the left leg length and the right leg length and may compare the calculated difference of leg lengths with a preset allowable imbalance range.

Here, the allowable imbalance range may be settings for determining whether the length difference between the left leg length and the right leg length is present in a normal range. This is information predetermined according to a professional opinion and the allowable imbalance range, as the normal range of the difference between the left and right leg lengths may be preset and stored in designing the massage device 1000.

When the imbalance determination portion 1218 determines the difference between the left and right leg lengths of the user to correspond to the allowable imbalance range, the leg length determination portion 1212 may calculate an average value between the left leg length and the right leg length and may determine the average value to be the user's leg length.

Meanwhile, when the imbalance determination portion 1218 determines the difference between the left and right leg lengths of the user as not corresponding to the allowable imbalance range, the leg massage portion 300 may be controlled while being separated into a left side and a right side according to the left leg length and the right leg length. To this end, the leg massage portion 300 may be provided to have a structure divided into a left side and a right side to be separately controllable. Accordingly, the massage device 1000 may separate and determine at least one of a position and an extension length of the leg massage portion 300 and a position of the upper leg module 320 into a left side and a right side according to the left leg length and the right leg length.

This may be a component configured to precisely adjust the growth plate stimulating massage pattern according to physical conditions of the user in consideration of a case in which the left leg length and the right leg length differ from each other due to a pelvic distortion, scoliosis, bowlegs, X-shaped curvature of the legs, or the like.

The height determination portion 1214 may obtain a shoulder height of the user through a sensing means and may determine the user's height on the basis of the shoulder height and the leg length of the user determined by the leg length determination portion 1212.

For example, when a longitudinal shoulder position of the user is measured through the sensing means, since length information of the body massage portion 100 is already known, the height determination portion 1214 may determine an upper body length of the user on the basis thereof. Also, a height of the user may be determined on the basis of the leg length and the upper body length of the user determined by the leg massage portion 300.

Here, in determining the height of the user, it is possible to further use stored information obtained by previously numerating information on a relationship between heights measured while a plurality of people stand upright and heights measured while the plurality of people are seated in the massage device 1000.

Here, the sensing means may include at least one of a graduated ruler, an infrared sensor, a touch sensor, a pressure sensor, an infrared sensor, and an optical sensor.

The upper-lower body proportion calculation portion 1216 may determine the upper-lower body proportion of the user on the basis of the height and the leg length of the user. This is a component configured to adjust the growth plate stimulating massage pattern in consideration of the upper-lower body proportion of the user and will be described below in detail.

The growth plate stimulating massage pattern determination portion 1230 may adjust the massage device 1000 according to the physical conditions of the user and may determine the growth plate stimulating massage pattern before providing the growth plate stimulating massage pattern.

To this end, the growth plate stimulating massage pattern determination portion 1230 may determine positions of the shoulder air cell 120 and he leg massage portion 300 on the basis of the shoulder position and the heel position of the user first, may adjust the position of the upper leg module 320 on the basis of the leg length of the user, and then may set an extension length of the leg massage portion 300 according to the leg length of the user.

Hereinafter, this will be described in detail with reference to the drawings.

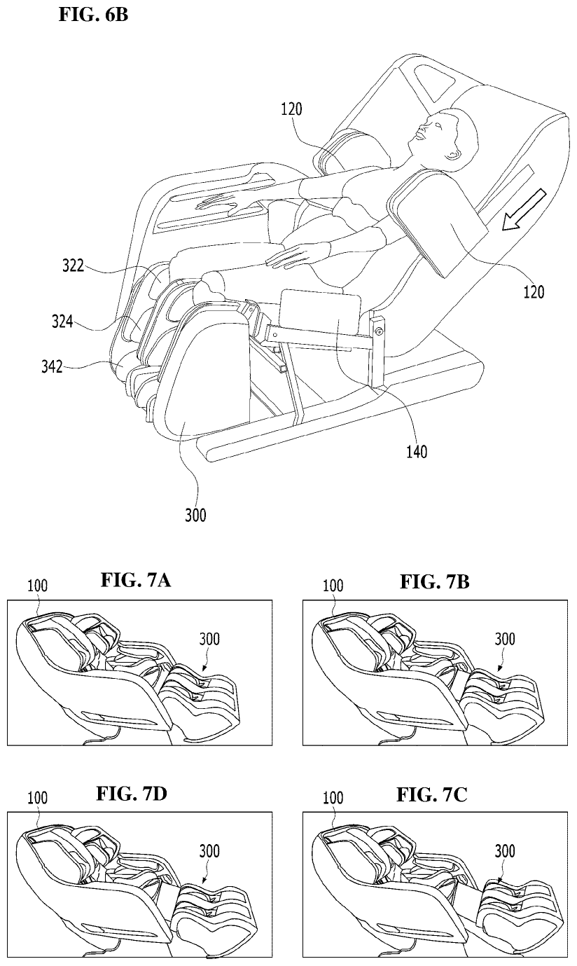

FIG. 5 illustrates a guide and a slide 122 provided below the shoulder air cell 120 according to one embodiment of the present disclosure, and FIGS. 6A and 6B illustrate one example in which the position of the shoulder air cell 120 according to one embodiment of the present disclosure is adjusted according to the physical conditions of the user.

The growth plate stimulating massage pattern determination portion 1230 may determine the position of the shoulder air cell 120 on the basis of the shoulder height of the user.

To this end, as shown in FIG. 5, the guide and the slide 122 configured to be vertically movable along the guide may be provided on one side of the body massage portion 100.

The shoulder air cell 120 may change in position by moving vertically according to the shoulder height of the user through the slide 122 moving vertically along the guide and may fix the position of the shoulder air cell 120 while providing a massage.

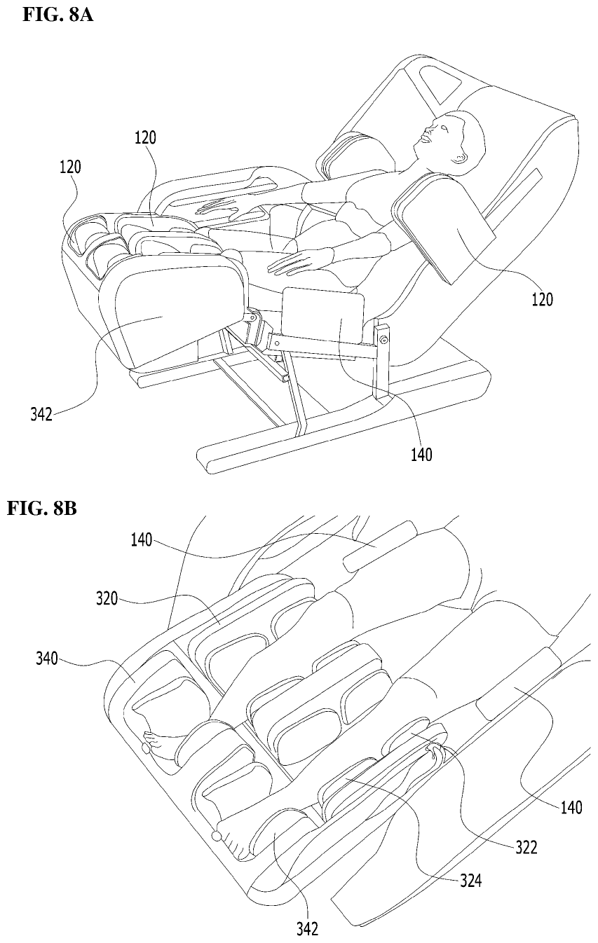

Also, the growth plate stimulating massage pattern determination portion 1230 may determine the position of the leg massage portion 300 on the basis of the heel position of the user. FIGS. 7A to 7D are exemplary views illustrating a method of determining the position of the leg massage portion 300 according to one embodiment of the present disclosure.

In order to determine the position of the leg massage portion 300 according to the leg length of the user, the leg massage portion 300 may extend with a maximum length in a longitudinal direction of the legs first and then may move in an opposite direction until the heels of the user are detected.

In other words, the leg massage portion 300 may extend by a maximum extendable length in the longitudinal direction of the legs and may gradually contract in a direction opposite to the longitudinal direction of the legs. Here, when the heels of the user are detected by a sensor while the leg massage portion 300 contracts, the leg massage portion 300 may stop contracting and moving. Accordingly, the position of the leg massage portion 300 may be determined. FIG. 7D illustrates a state in which the leg massage portion 300 extends to the maximum length. From FIG. 7A toward FIG. 7C, the position of the leg massage portion 300 extends in the longitudinal direction of the legs.

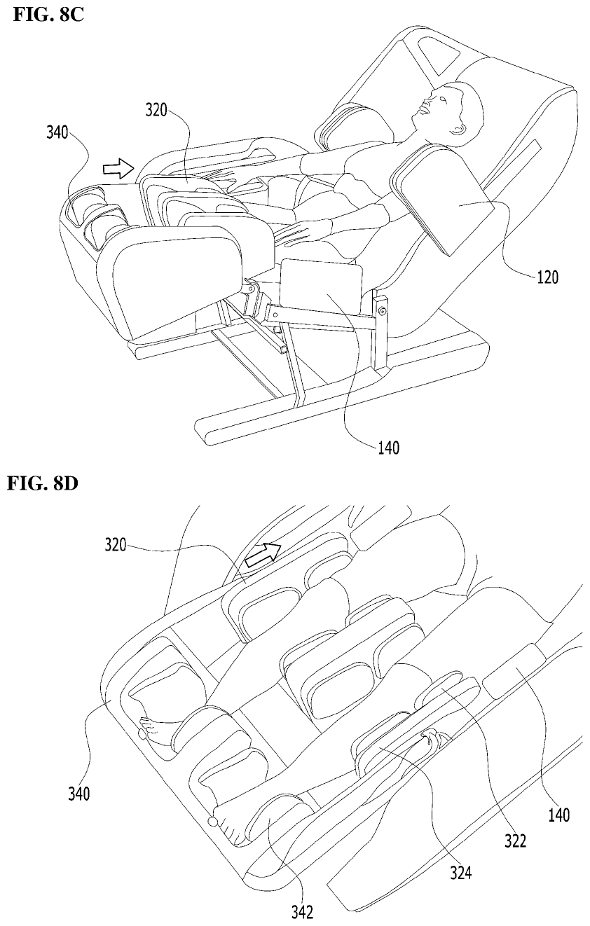

FIGS. 8A and 8B illustrate one example in which the position of the upper leg module 320 according to one embodiment of the present disclosure is adjusted on the basis of physical conditions of the user.

The growth plate stimulating massage pattern determination portion 1230 may determine the position of the upper leg module 320 in a state in which the position of the leg massage portion 300 is determined using the above method.

To this end, the growth plate stimulating massage pattern determination portion 1230 may determine a range to which the height and leg length of the user correspond on the basis of a standard range of leg length set differentially according to a height. Also, the position of the upper leg module 320 may be determined according to the determined range.

For example, when a standard leg length is 92 to 93 cm in a case that a height is 160 cm, when the height of the user is 160 cm and the leg length is 92 cm or less, the leg length of the user may be determined to be within a range smaller than or equal to the standard range. Here, the standard range of leg length may be set differentially according to each height band and may be prestored.

When the leg length of the user is determined to be within the range smaller than or equal to the standard range, on the basis of a first position of the upper leg module 320 which is preset according to the standard range as shown in FIG. 8A, the growth plate stimulating massage pattern determination portion 1230 may determine the position of the upper leg module 320 to be lower than the first position.

Since a knee part of a short leg is relatively lower than that of a long leg, it is necessary to determine a position of the upper air cell 322 to be relatively low.

On the other hand, when the leg length of the user is determined to be within the range greater than or equal to the standard range, on the basis of the first position of the upper leg module 320 which is preset according to the standard range as shown in FIG. 8C, the position of the upper leg module 320 may be determined to be higher than the first position.

The position of the upper air cell 322 may be set on positions of both sides of the bottom end of the femur of the user using the above method.

As shown in FIGS. 8B and 8D, the upper air cell 322 may expand to fix both sides of the bottom end of the femur of the user.

Also, the growth plate stimulating massage pattern determination portion 1230 may determine an extension length of the leg massage portion 300 on the basis of the leg length of the user. Here, the extension length may mean a length of elongating the leg massage portion 300 in an operation of extending the leg massage portion 300 downward to stimulate a growth plate of a knee while the growth plate stimulating massage pattern is provided.

For example, when an extension length of the lower leg module 340 is determined to be 5 cm with respect to a person who has a leg length of 90 cm, the extension length of the lower leg module 340 may be determined to be 3.88 cm with respect to a person who has a leg length of 70 cm.

As another embodiment, the growth plate stimulating massage pattern determination portion 1230 may adjust the massage device 1000 in consideration of the user's height.

In detail, the growth plate stimulating massage pattern determination portion 1230 may determine at least one of an angle variation degree between the leg massage portion 300 and the body massage portion 100 according to the height of the user, a depth level of pushing, by the massage module 1700 corresponding to a spine part, the spine, and a time for injecting air into the air cells.

To this end, the growth plate stimulating massage pattern determination portion 1230 may determine a height level to which the user's height corresponds on the basis of height levels divided into certain levels on the basis of a certain height.

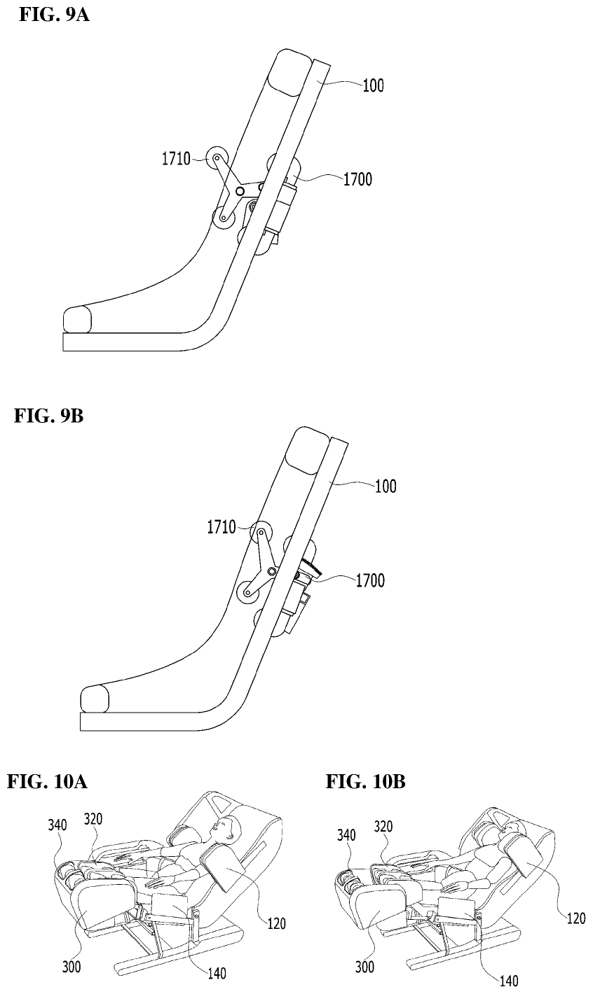

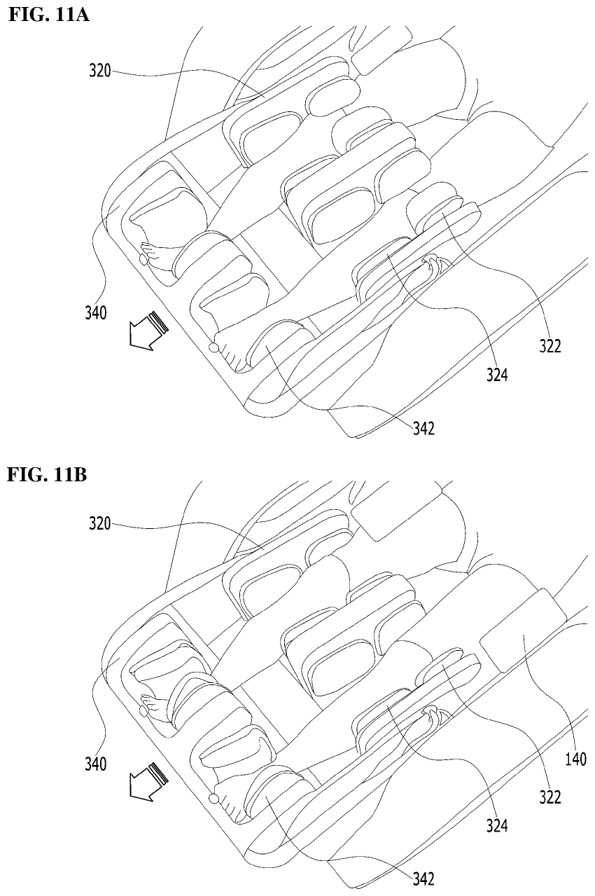

In detail, when the user's height is determined to correspond to a height level lower than or equal to the certain height, on the basis of a first reference depth range preset with respect to a depth degree of pushing, by the massage module 1700, the spin according to the certain height, the depth degree of pushing, by the massage module 1700, the spine may be determined to be within a depth range shallower than the first reference depth range. FIGS. 9A and 9B illustrate one example of adjusting a depth degree to which the massage module 1700 protrudes to push the spine.

Also, when the user's height is determined to correspond to the height level lower than or equal to the certain height, one the basis of a first reference injection time preset with respect to a time of injecting air into each air cell according to the certain height, the growth plate stimulating massage pattern determination portion 1230 may determine the time of injecting air into each air cell to be shorter than the first reference injection time. Here, the air cells may mean air cells related to the growth plate stimulating massage pattern, that is, the shoulder air cell 120, a pelvis air cell 140, the upper air cell 322, an intermediate air cell 324, and the lower air cell 342.

Also, when the user's height is determined to correspond to the height level lower than or equal to the certain height, on the basis of a first reference angle range preset with respect to an angle variation degree between the leg massage portion 300 and the body massage portion 100 according to the certain height, the growth plate stimulating massage pattern determination portion 1230 may determine the angle variation degree between the leg massage portion 300 and the body massage portion 100 to be within an angle range narrower than the first reference angle range.

Here, for example, a change in an angle between the leg massage portion 300 and the body massage portion 100 may be included operations of the massage device 1000 for providing the growth plate stimulating massage pattern.

On the other hand, when the user's height is determined to correspond to a height level higher than or equal to the certain height, on the basis of the first reference depth range preset with respect to the depth degree of pushing, by the massage module 1700, the spin according to the certain height, the depth degree of pushing, by the massage module 1700, the spine may be determined to be within a depth range deeper than the first reference depth range.

Also, when the user's height is determined to correspond to the height level higher than or equal to the certain height, on the basis of a first reference injection time preset with respect to a time of injecting air into each air cell according to the certain height, the growth plate stimulating massage pattern determination portion 1230 may determine the time of injecting air into each air cell to be longer than the first reference injection time. Here, the air cells may mean air cells related to the growth plate stimulating massage pattern, that is, the shoulder air cell 120, the pelvis air cell 140, the upper air cell 322, the intermediate air cell 324, and the lower air cell 342.

Also, when the user's height is determined to correspond to the height level higher than or equal to the certain height, on the basis of a first reference angle range preset with respect to an angle variation degree between the leg massage portion 300 and the body massage portion 100 according to the certain height, the growth plate stimulating massage pattern determination portion 1230 may determine the angle variation degree between the leg massage portion 300 and the body massage portion 100 to be within an angle range greater than the first reference angle range.

As another embodiment, the growth plate stimulating massage pattern determination portion 1230 may adjust the massage device 1000 on the basis of the upper-lower body proportion of the user.

In detail, the growth plate stimulating massage pattern determination portion 1230 may determine a range to which the height and upper-lower body proportion of the user correspond on the basis of a standard range of upper-lower body proportion set differentially according to the user's height. Also, at least one of the depth degree of pushing, by the massage module 1700 corresponding to the spine part, the spine and the time of injecting air into the air cell may be determined according to the determined range. Here, the air cell may mean the air cells related to the growth plate stimulating massage pattern.

For example, when a lower body proportion of the user is determined to be a short range with respect to the upper-lower body proportion of the user on the basis of the standard range, on the basis of a second reference depth range preset according to the standard range with respect to the depth degree of pushing, by the massage module 1700, the spine, the depth degree of pushing, by the massage module 1700, the spine may be determined to be a range deeper than the second reference depth range.

Here, the second reference depth range may be calculated in consideration of the depth degree determined on the basis of the first reference depth range according to the above user's height. In other words, a protruding degree of the massage module 1700 may be determined primarily on the basis of the user' height and then the protruding degree of the massage module 1700 may be determined secondarily on the basis of the upper-lower body proportion of the user.

Also, when the lower body proportion of the user is determined to be within a short range on the basis of the standard range with respect to the upper-lower body proportion of the user, on the basis of a second reference injection time preset according to the standard range with respect to the time of injecting air into the air cell, the time of injecting air into the air cell may be determined to be longer than the second reference injection time.

Here, the second reference injection time may be calculated in consideration of the injection time determined on the basis of the first reference injection time according to the above user's height. In other words, the time of injecting air into the air cell may be determined primarily on the basis of the user's height and then the time of injecting air into the air cell may be adjusted secondarily on the basis of the upper-lower body proportion of the user.

As described above, since the massage device according to embodiments of the present disclosure multiply considers two or more physical conditions in addition to a single physical condition, it is possible to reflect more detailed physical conditions of the user in controlling the massage device.

In addition, since the massage device according to embodiments of the present disclosure may implement adjustment stepwise according to a variety of physical conditions with respect to one component of the device, it is possible to implement a massage device more optimized to the user.

Accordingly, the massage device according to embodiments of the present disclosure may provide a massage pattern stimulating a growth plate to promote growth of growing children or adolescents who have a height of about 120 to 160 cm.

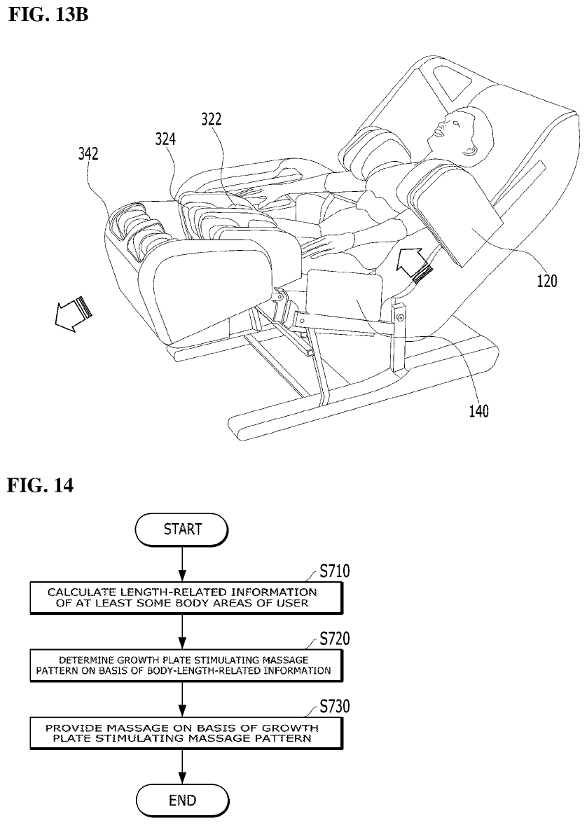

FIGS. 11A and 13B are exemplary views illustrating the grow plate stimulating massage pattern according to embodiments of the present disclosure.

The growth plate stimulating massage pattern according to embodiments of the present disclosure may include a knee growth plate stimulating massage pattern, a spine growth plate stimulating massage pattern, and a knee and spine growth plate stimulating massage pattern.

Referring to FIG. 11A, in a knee growth plate stimulating massage pattern according to a first embodiment, while air for fixing both side parts of the bottom end of the femur may be supplied to the upper air cell 322 and air for fixing an ankle area may be supplied to the lower air cell 342, the lower leg module 340 may extend downward.

Here, the lower leg module 340 may not only extend downward but also stimulate a growth plate of a knee area of the user by generating an axial pressure with respect to the knee area of the user while alternately moving upward and downward. Here, alternate movement of the lower leg module 340 upward and downward may be repetitively performed in a certain cycle for a certain time.

The knee growth plate stimulating massage pattern according to the first embodiment may provide a massage stimulating the growth plate of the knee area by stretching the legs while an upper knee area and the ankle area are fixed.

Referring to FIG. 11B, in a knee growth plate stimulating massage pattern according to a second embodiment, air for fixing a pelvic area may be supplied to the pelvis air cell 140, air for fixing the ankle area may be supplied to the lower air cell 342, and the lower leg module 340 may extend downward.

Here, an axial pressure may be generated at a leg area by repetitively extending the lower leg module 340 downward and then contracting the lower leg module 340 upward.

The knee growth plate stimulating massage pattern according to the second embodiment may provide a massage stimulating the growth plate of the knee area by stretching an entirety of the legs while a pelvis area and the ankle area are fixed.

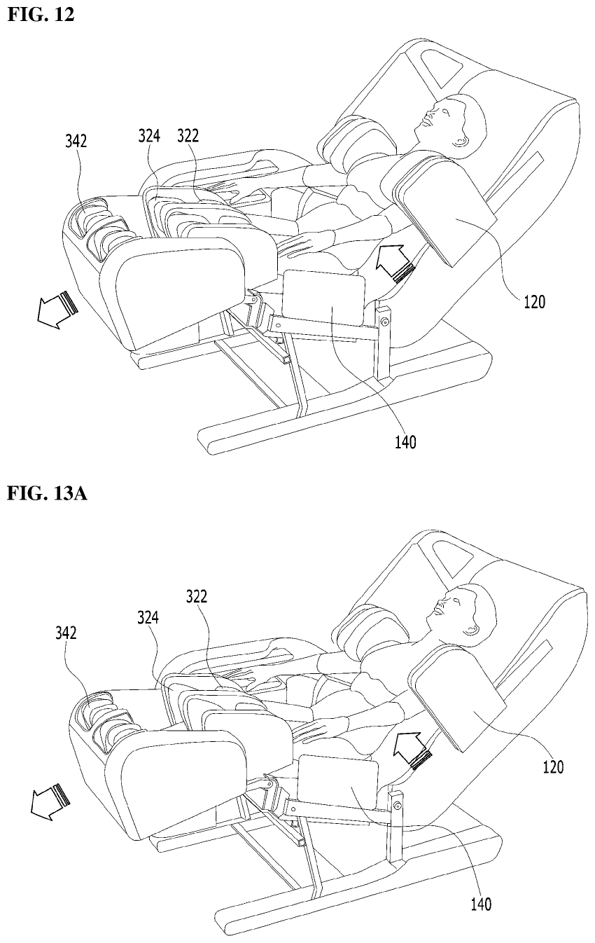

Referring to FIG. 12, in the spine growth plate stimulating massage pattern according to embodiments of the present disclosure, air for fixing a shoulder area may be supplied to the shoulder air cell 120, air for fixing the leg area may be supplied to the upper air cell 322, the intermediate air cell 324, and the lower air cell 342, the massage module 1700 may protrude, and the leg massage portion 300 may extend downward.

The spine growth plate stimulating massage pattern may provide a massage of stretching a torso and stimulating a growth plate of the spine area by fixing the shoulder area and the leg area, extending the spine area, and stretching the legs.

Referring to FIG. 13A, in a spine and knee growth plate stimulating massage pattern according to the first embodiment, air for fixing the shoulder area may be supplied to the shoulder air cell 120 and air for fixing the ankle area may be supplied to the lower air cell 342, the massage module 1700 may protrude, and the lower leg module 340 may extend downward.

The spine and knee growth plate stimulating massage pattern according to the first embodiment may provide a massage of stretching the torso and the legs and stimulating the growth plate of the spine area and the growth plate of the knee area by fixing the shoulder area and the ankle area, extending the spine area, and stretching the legs.

Referring to FIG. 13B, in a spine and knee growth plate stimulating massage pattern according to the second embodiment, air for fixing the shoulder area may be supplied to the shoulder air cell 120, and air for fixing a part outside a nasal bone and a part outside a neck bone may be supplied to the intermediate air cell 324, and air for fixing the ankle area may be supplied to the lower air cell 342, the massage module 1700 may protrude, and the lower leg module 340 may extend downward.

The spine and knee growth plate stimulating massage pattern according to the second embodiment may provide a massage of stretching the torso and stimulating the growth plate of the spine area and the growth plate of the knee area by fixing the shoulder area, the ankle area, the part outside nasal bone, and the part outside the neck bone, extending the spine area, and stretching the legs.

In the above growth plate stimulating massage pattern, an air injection time of supplying air to the air cell, a depth degree of pushing, by the massage module 1700, the spine, a variation degree of an angle between the leg massage portion 300 and the body massage portion 100, an extension length of the lower leg module 340, and a position of the upper leg module 320 may be based on determination of the growth plate stimulating massage pattern determination portion 1230 as described above.

Also, the massage device 1000 according to embodiments of the present disclosure may provide a brain massage as well as the growth plate stimulating massage promoting growth.

The brain massage may mean an operation of inducing a stimulus for a brainwave of a person to be massaged by transmitting an acoustic signal having an arbitrary form such as a binaural beat to the person to be massaged so as to improve concentration of the person to be massaged, to relieve stress, or the like. Here, the acoustic signal may be provided through the audio output module 1600. A sound source having an arbitrary form obtained by mixing the binaural beat with an arbitrary type of music including classic music, pop music, and the like may be used for a brain massage. The binaural beat means a particular type of audio information capable of adjusting a brainwave. A detailed method of mixing a binaural beat with music may refer to a technique disclosed in Korean Patent Registration No. 10-1858927.

FIG. 14 is a flowchart illustrating a method of providing the growth plate stimulating massage promoting growth according to embodiments of the present disclosure.

In operation S710, the massage device 1000 may calculate length-related information of at least some body areas of a user. A method of calculating, by the massage device 1000, the length-related information of at least some body areas of the user has been described in detail above with reference to FIG. 3.

In operation S720, the massage device 1000 may determine a growth plate stimulating massage pattern on the basis of body-length-related information. A method of determining, by the massage device 1000, the growth plate stimulating massage pattern has been described above in detail with reference to FIG. 3.

In operation S730, the massage device 1000 may provide the user with a massage on the basis of the determined growth plate stimulating massage pattern. A method of providing, by the massage device 1000, the user with the massage on the basis of the growth plate stimulating massage pattern has been described above in detail with reference to FIGS. 11A to 13B.

It may be well understood by those skilled in the art that the present invention may be implemented by coupling other program modules and/or a combination of hardware and software. For example, the present invention may be implemented by a computer-readable medium.

Any computer-accessible medium may be the computer-readable medium, and the computer-readable medium includes volatile and nonvolatile media, transitory and non-transitory media, and portable and nonportable media. As a nonrestrictive example, the computer-readable medium may include a computer-readable storage medium and a computer-readable transmission medium.

The computer-readable storage medium may include volatile and nonvolatile media, transitory and non-transitory media, and portable and nonportable media implemented by an arbitrary method or technique of storing information such as a computer-readable command, a data structure, a program module, or other data. The computer-readable storage medium includes an RAM, a read-only memory (ROM), an electrically erasable and programmable ROM (EEPROM), a flash memory, or other memory techniques, a compact disc-ROM (CD-ROM), a digital video disk (DVD), or other optical disks, a magnetic cassette player, a magnetic tape, a magnetic disk storage device, other magnetic storage device, or random other media accessible by a computer and used for storing desired information but is not limited thereto.

The computer-readable transmission medium includes all information transmission media configured to implement a computer-readable command, a data structure, a program module, other data, or the like generally in a modulated data signal such as a carrier wave and other transport mechanism. The term “modulated data signal” means a signal with one or more features set or changed to encode information in the signal. As a nonrestrictive example, the computer-readable transmission medium includes a wired medium such as a wired network or a direct-wired connection and a wireless medium such as a sound, a radio frequency, an infrared ray, and other wireless media. It should be noted that a combination of arbitrary ones of the above-described media may be included in the computer-readable transmission medium.

It should be understood by one of ordinary skill in the art that a variety of exemplary logics, blocks, modules, processors, means, circuits, and algorithm steps which have been described in relation to the embodiments disclosed herein may be implemented by electronic hardware, a variety of types of programs, design codes, and a combination thereof (for convenience, referred herein to as “software”). In order to definitely explain such interoperability between hardware and software, a variety of exemplary components, blocks, modules, circuits, and operations have been generally described in relation to functions thereof. Whether the functions are implemented by hardware and software depends on design limitations imposed on a particular application and the entire system. Although one of ordinary skill in the art can implement functions described in a variety of ways with respect to each particular application, it should be construed that the implementations do not depart from the scope of the present invention.

A variety of embodiments disclosed herein may be implemented by a manufactured article using a method, device, or a standard programming and/or engineering technique. The term “manufactured article” includes a computer program, carrier, or media accessible by an arbitrary computer-readable device. For example, the computer-readable storage medium includes a magnetic storage device (for example, a hard disk, a floppy disk, a magnetic strip, and the like), an optical disk (for example, a compact disc (CD), DVD, and the like), a smart card, and a flash memory device (for example, an EEPROM, a card, a stick, a key drive, and the like) but is not limited thereto. The term “machine-readable medium” includes a wireless channel and a variety of other media capable of storing, containing, and/or transmitting command(s) and/or data but is not limited thereto.

It should be noted that a particular sequence or hierarchy of operations in the disclosed processes are merely examples of exemplary accesses. It should be understood that the particular sequence or hierarchy of the operations in the processes may be rearranged without departing from the scope of the present invention on the basis of design priorities. The attached method-related claims provide elements of a variety of operations as a sample sequence but are not limited to the disclosed particular sequence or hierarchy.

The description of the disclosed embodiments is provided to allow one of ordinary skill in the art to utilize or perform the present invention. A variety of modifications of the embodiments are apparent to one of ordinary skill in the art, and general principles defined herein are applicable to other embodiments without departing from the scope of the present invention. Therefore, the present invention should not be limited to the embodiments provided herein and should be construed within the broadest scope consistent to the principles and novel features provided herein.

1. A massage device configured to provide a growth plate stimulating massage promoting growth, comprising:
a body data calculation portion configured to calculate length-related information of at least some body areas of a user;
a growth plate stimulating massage pattern determination portion configured to determine a growth plate stimulating massage pattern on the basis of the calculated body-length-related information; and
a growth plate stimulating massage providing portion configured to provide a massage on the basis of the growth plate stimulating massage pattern.

2. The massage device of claim 1, comprising:
a body massage portion comprising at least one air cell and a massage module; and
a leg massage portion comprising an upper air cell, an intermediate air cell, and a lower air cell,
wherein the leg massage portion has a structure divided into an upper leg module and a lower leg module to be vertically movable independently.

3. The massage device of claim 2, wherein the body data calculation portion comprises:
a leg length determination portion configured to obtain a heel position of the user and to determine a leg length on the basis thereof; and
a height determination portion configured to obtain a shoulder height of the user and to determine a height of the user on the basis of the leg length and the shoulder height, and
wherein the growth plate stimulating massage pattern determination portion controls at least a part of the massage device on the basis of at least one of the shoulder height, leg length, and height of the user.

4. The massage device of claim 3, wherein the growth plate stimulating massage pattern determination portion determines a position of the shoulder air cell according to the shoulder height of the user and determines a position of the leg massage portion according to the leg length of the user.

5. The massage device of claim 4, wherein on the basis of a standard range of leg length set differentially according to a height, the growth plate stimulating massage pattern determination portion determines a range to which the height and leg length of the user correspond on the basis of the standard range and determines a position of the upper leg module according to the determined range.

6. The massage device of claim 5, wherein when the leg length of the user is determined to be within a range smaller than or equal to the standard range, on the basis of a first position of the upper leg module preset according to the standard range, the growth plate stimulating massage pattern determination portion determines the position of the upper leg module to be lower than the first position.

7. The massage device of claim 3, wherein the growth plate stimulating massage pattern determination portion determines an extension length of the lower leg module on the basis of the leg length of the user.

8. The massage device of claim 3, wherein the growth plate stimulating massage pattern determination portion determines a height level to which the height of the user corresponds on the basis of height levels divided into certain levels on the basis of a certain height and determines at least one of a variation degree of an angle between the leg massage portion and the body massage portion, a depth degree of pushing, by the massage module corresponding to a spine area, a spine, and a time for injecting air into the air cell according to the determined height level.

9. The massage device of claim 8, wherein when the height of the user is determined to be a height level smaller than the certain height, the growth plate stimulating massage pattern determination portion performs at least one of:
determining, on the basis of a first reference depth range preset according to the certain height, the depth degree of pushing, by the massage module, the spine to be a depth range shallower than the first reference depth range; and
determining, on the basis of a first reference injection time preset according to the certain height, the time for injecting air into the air cell to be shorter than the first reference injection time.

10. A method of providing a growth plate stimulating massage promoting growth, comprising:
calculating length-related information of at least some body areas of a user;
determining a growth plate stimulating massage pattern on the basis of the calculated body-length-related information; and
providing a massage on the basis of the growth plate stimulating massage pattern.
